(19) United States
(12) Patent Application Publication
(10) Pub. No.: US 2025/0090719 A1
(43) Pub. Date: Mar. 20, 2025

(54) BIOMATERIALS FOR EMBOLIZATION AND DRUG DELIVERY

(71) Applicant: Regents of the University of Minnesota, Minneapolis, MN (US)

(72) Inventors: Jafar Golzarian, Plymouth, MN (US); Chen Guo, Edina, MN (US)

(21) Appl. No.: 18/291,525

(22) PCT Filed: Jul. 29, 2022
(86) PCT No.: PCT/US2022/074308

§ 371 Date: Jan. 23, 2024

Related U.S. Application Data
(60) Provisional application No. 63/227,769, filed on Jul. 30, 2021.

(51) Int. Cl. A61L 24/10; A61L 24/00; A61L 24/04

(52) U.S. Cl. CPC A61L 24/108; A61L 24/0031; A61L 24/046; A61L 2400/06; A61L 2430/36

(57) ABSTRACT

A biomaterial includes keratin proteins crosslinked with linking groups chosen from thiosuccinimide, thioether sulfone, and mixtures and combinations thereof.

This application is a PCT application that claims priority to and the benefit of U.S. Provisional Patent Application No. 63/227,769, filed Jul. 30, 2021, the entire contents of which is incorporated herein by reference.

### BACKGROUND

Therapeutic embolization is the intentional endovascular occlusion of an artery or vein. The embolic agent of choice depends on the desired clinical outcome, as well as the inherent properties and behavior of the agent. Suitable embolic agents may be temporary or permanent. Example embolic agents include liquids such as collagen, thrombin, gelatin foam, polyvinyl alcohol (PVA), glues such as cyanoacrylates, the liquid embolic system available under the trade designation ONYX from Medtronic, Inc., Minneapolis, MN, other liquid embolic agents such as Squid and PHIL, as well as solid vascular occlusion devices such as coils, plugs, balloons, and the like.

### SUMMARY

In one aspect, the present disclosure is directed to biomaterials including keratin proteins. The keratin proteins are crosslinked to form linking groups chosen from thiosuccinimide, thioether sulfone, and mixtures and combinations thereof. In some examples, the thiol-containing keratin proteins form hydrogels that can be used in a wide variety of applications including, but not limited to, embolic agents to occlude vasculature of the human body, agents for drug delivery, or scaffolds for cell growth. In some examples, the thiol-reactive crosslinks are functional or multifunctional, and can include oligomeric or polymeric functional groups such as, for example, water soluble synthetic polymers and biopolymers including alkylene oxide oligomers such as poly (ethylene glycol) (PEG), which may itself optionally be functionalized, a functional or non-functional polysaccharide and polypeptide, and the like. As described herein, the embolic agents may be in liquid form, particles suspended in a liquid, or solid forms.

In some examples, the keratin proteins in the hydrogels of the present disclosure are obtained from human hair. When used as an embolic agent in the human body, the hydrogels have excellent biocompatibility and would be expected to have fewer issues of immunogenicity compared to embolic agents derived from synthetic or animal-based materials.

The hydrogels of the present disclosure form rapidly (e.g., less than 10 minutes, less than 1 minutes, or even less than 10 seconds) in aqueous solution, and no potentially toxic organic solvents such as DMSO are required for crosslinking. In various examples, the hydrogels form via thiol-maleimide or thiol-vinylsulfone addition reactions that may be conducted without catalysts or application of radiation (for example, ultraviolet light (UV)) over a wide range of temperatures, and react cleanly to produce little or no potentially harmful byproducts. Multiple linking groups from these reactions can enable the crosslinking to occur which results in example hydrogels herein. The properties of the hydrogels of the present disclosure can be easily varied by adjusting reactant concentration. For example, in some examples the hydrogel gelation time is sufficiently fast for clinical applications, and the gelation time is readily tunable depending on the intended application. The reactants used to form the hydrogels have low viscosities, and can be administered to a patient via single or dual lumen microcatheters, which make possible precisely selected embolization. Once formed, the hydrogels may be readily degraded as needed. In some examples, as described herein, the hydrogels may be constructed to be dissolvable or rapidly dissolvable hydrogels that can act as keratin embolic agents. Generally the hydrogels described herein may be dissolved in less than 10 minutes. However, rapidly dissolvable hydrogels may be dissolvable in less than 5 minutes or less than 3 minutes, for example. In this manner, the hydrogel may be dissolved to revert the embolization without damaging tissue if needed.

In some examples, the hydrogels may be loaded with one or more biological or therapeutic agents such as, for example, drugs like small molecule drugs or protein drugs.

In one aspect, the present disclosure is directed to a biomaterial including keratin proteins crosslinked with linking groups chosen from thiosuccinimide, thioether sulfone, and mixtures and combinations thereof.

In another aspect, a method for making the biomaterial includes reacting a first aqueous pre-gel solution comprising a water soluble keratein with an aqueous solution comprising a crosslinking compound chosen from maleimide, vinyl sulfone, and mixtures and combinations thereof, to create the biomaterial comprising the keratin proteins crosslinked with at least one of the thiosuccinimide or the thioether sulfone.

In another aspect, a system for delivering components that react to form the biomaterial includes at least one catheter configured to be disposed within a blood vessel, and wherein the at least one catheter is configured to deliver the components that react to form, in the blood vessel and outside of the at least one catheter, the keratin proteins crosslinked with at least one of the thiosuccinimide or the thioether sulfone.

In another aspect, the present disclosure is directed to a hydrogel including a biomaterial in an aqueous medium, wherein the biomaterial includes keratin proteins crosslinked with thiosuccinimide linking groups.

In another aspect, the present disclosure is directed to a method for making a hydrogel, the method including reacting a first aqueous pre-gel solution including a water soluble keratein with a second aqueous solution including a crosslinking compound chosen from maleimide, vinyl sulfone, and mixtures and combinations thereof.

In another aspect, the present disclosure is directed to a method for occluding a blood vessel, the method including introducing into the blood vessel an embolic agent having keratin proteins crosslinked with linking groups chosen from thiosuccinimide, thioether sulfone, and mixtures and combinations thereof.

In another aspect, the present disclosure is directed to a kit for conducting an embolization procedure. The kit includes a first powdered water soluble keratein; a crosslinker with a maleimide or vinylsulfone compound; and at least one catheter configured to deliver the first aqueous solution, the keratein, and the crosslinker to an occlusion site to form a hydrogel at the occlusion site.

The details of one or more examples of the invention are set forth in the accompanying drawings and the description below. Other features, objects, and advantages of the invention will be apparent from the description and drawings, and from the claims.

### BRIEF DESCRIPTION OF DRAWINGS

FIG. 1 is schematic of oxidation and reduction schemes for keratin.

FIG. 2 is a schematic representation of reactions in which disulfide bonds in keratin are converted to thiol groups as the keratein was chemically reduced and extracted from human hair.

FIG. 3A is a schematic representation of reactions in which the thiol groups on the keratein of FIG. 2 is reacted with functional groups from functionalized crosslinking compounds to form covalent bonds.

FIG. 3B is a schematic representation of example rapidly dissolvable biomaterials including keratin.

FIGS. 3C and 3D are a schematic representations of example reactions in which the thiol groups on the keratein of FIG. 2 is reacted with functional groups from functionalized crosslinking compounds to form covalent bonds.

FIG. 4 is a schematic representation of a reaction between a multifunctional maleimide crosslinker and the keratein of FIG. 2 to form a crosslinked hydrogel.

FIG. SA is a schematic representation of a dual catheter apparatus that may be used to apply the crosslinked hydrogels of the present disclosure as embolic agents to occlude an artery or a vein.

FIGS. 5B, 5C, and 5D are conceptual diagrams of example catheters configured to deliver different materials to form embolic agents to occlude a blood vessel.

FIGS. 5E and 5F are conceptual diagrams illustrating a method for delivering different components of the present disclosure at different locations within a blood vessel to form embolic agents.

FIGS. 6A-6E are plots showing the change in gelation time as the concentration of keratein and PEG in a pre-gelling solution and the molecular weight and shape and number of functionality of maleimide crosslinkers are varied.

FIGS. 7A-7B are plots showing the change in modulus when an additive such as a contrast dye is incorporated into the pre-gelling solutions of Example 2.

FIGS. 8A-8B are plots showing the swelling ratio of various crosslinked hydrogels in Example 3 following overnight storage.

Like symbols in the drawings indicate like elements.

### DETAILED DESCRIPTION

In general, therapeutic embolization is the intentional endovascular occlusion of an artery or vein, which may be temporary or permanent. Liquid embolic agents can have advantages over solid embolic agents including, for example, more complete and efficient filling, particularly when the geometry of the vessel includes irregularities. However, commercially available liquid embolic agents such as cyanoacrylates and ONYX can suffer many drawbacks including difficult administration, off-target embolization, and the require the use of cytotoxic organic solvents. For example, ONYX polymers precipitate from relatively non-biocompatible solvents such as dimethylsulfoxide (DMSO) to blood, and require special catheters and syringes that can be inconvenient and costly to use in certain clinical applications.

Keratin is a cysteine-rich intracellular cytoskeleton protein, which is readily available from animal hair including wool, chicken feathers and human hair. Keratin and its derivatives are inexpensive, have excellent biocompatibility, are possibly biodegradable, are hemostatic, and are less likely to provoke adverse immune reactions when used in or on the human body. In some examples, keratin hydrogels have been used in wound dressing, hemostatic dressings, and tissue regeneration. However, these keratin hydrogels are formed through physical interactions and disulfide bonds, and their formation reactions are time consuming or require the use of oxidizing agents. The reaction may take hours or even days to complete. Therefore, these hydrogels are not well suited for use as embolic agents in a patient, which in many cases require rapid in-situ formation in a vessel using biocompatible reactants and solvents. Liquid embolic agents are thus needed that are simple to administer, biocompatible and precisely deliverable.

As discussed above, keratins are a family of proteins found in the hair, skin, and other tissues of vertebrates. Hair is a unique source of human keratins because it is one of the few human tissues that are readily available and inexpensive. Although other sources of keratins are acceptable feedstocks for the biomaterials of the present disclosure (e.g. wool, fur, horns, hooves, beaks, feathers, scales, and the like), human hair is preferred because of its biocompatibility in human medical applications.

As described herein, the present disclosure is generally directed to biomaterials including keratin proteins. The keratin proteins may be crosslinked with linking groups chosen from thiosuccinimide, thioether sulfone, and mixtures and combinations thereof. In some examples, the thiol-crosslinked keratin proteins form hydrogels that can be used in a wide variety of applications including, but not limited to, embolic agents to occlude vasculature of the human body, agents for drug delivery, or scaffolds for cell growth. In some examples, the linking groups formed using a thiol reaction may be referred to as thiol linking groups.

Keratin protein(s) as used herein collectively refers to keratin in keratin protein sources, including but not limited to naturally occurring keratin, reduced keratin, and/or oxidized keratin, or S-sulfonated keratin. This term also refers to the extracted keratin derivatives that are produced by oxidative and/or reductive treatment of keratin, including but not limited to keratose, alpha-keratose, gamma-keratose, kerateins, alpha-keratein, or gamma-keratein.

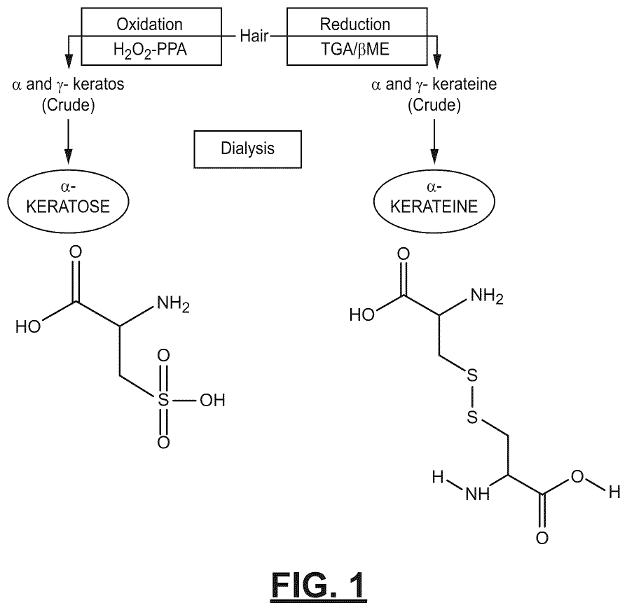

As shown schematically in the example of FIG. 1, keratins can be extracted from human hair fibers by oxidation or reduction processes. If a reductive treatment is used, the resulting materials are referred to as kerateins. In some examples, these reduction methods employ a process in which the hair is washed with detergent, water, and ethanol, de-lipidized using a compound such as chloroform or methanol, and then the crosslinked structure of the keratins is broken down by reduction.

In these reduction reactions, the disulfide bonds in the cystine amino acid residues are cleaved, rendering the keratins soluble without appreciable disruption of amide bonds. As shown schematically in FIG. 1, in some examples kerateins can be reduced by treating a keratin protein source with a reductant compound such as, for example, sodium sulfide, or thioglycolic acid (TGA) with beta-mercaptoethanol (βME), along with an optional base. In some examples, TGA is added to the keratin protein source at a ratio of about 5:1 to about 50:1, or about 25:1. The TGA is added at a solution ranging in concentrations from about 0.1 to about 10M, or about 0.5M. In some examples, the base is added to the drained keratin protein source in a ratio of about 10:1 to about 50:1, or at a concentration of about 100 mM. In other examples, sodium sulfide may be added to the keratin protein in similar concentrations as the TGA. In one example, 0.5M sodium sulfide may be used, but other concentrations may be used in different examples.

During extraction, mechanical agitation may optionally be used, and the keratein protein in the solution with base is mixed with agitation of at least 2 hours at 40° C. The solution containing the base and extracted keratin proteins (soluble keratin protein solution) may then be filtered to remove residual hair and stored.

The concentration may be modified to vary the degree of reduction, with concomitant alterations in pH, reaction time, temperature, and liquid to solid ratio. In some examples, the reduction is performed at a temperature between 0 and 100° C. for a reduction time of about 0.5 hours to about 24 hours, at a basic pH.

In some examples, residual reductant and denaturing agents can be removed from solution by dialysis. Typical dialysis conditions are 1% to 2% solution of kerateins dialyzed against purified water. In many instances during protein purification, dialysis is used to separate or even to concentrate certain protein species present in the sample. In some examples, the clarified protein solution is subjected to a dialysis step to fractionate certain protein species. In some examples, a 10 kDa molecular weight cutoff membrane is used to purify alpha-keratose or alpha-keratein. In other examples, a 5 kDa molecular weight cutoff membrane is employed to purify gamma-keratose or gamma keratein. Regenerated cellulose dialysis membranes may be used, however, many other membrane preparations suitable for protein purification are suitable.

In many instances, pressure is applied to aid in the dialysis process. If the pressure applied is too low, the resultant solutions contain greater protein fragments and peptides. Conversely, if the pressure is too high, the result is protein complex degradation. Thus, in some examples, the dialysis is performed under conditions that maintain a transmembrane pressure from about 30 to about 70 psi. Further, the heat buildup developed by the shear stress of pressurized dialysis is minimized by carrying out the dialysis at a temperature from about 4° C. to about 20° C. Additionally, as the solution is dialyzed, in some examples the conductivity is adjusted. In some examples, the conductivity is adjusted down to about or below 0.6 mS. In some instances, the conductivity is adjusted with water.

Those skilled in the art will recognize that other methods exist for the removal of low molecular weight contaminants in addition to dialysis (e.g. microfiltration, chromatography, and the like). In some examples, dissolving human hair with a solution, such as sodium sulfide, may dissolve the hair without providing acceptable amounts of available thiols for reactions. Therefore, the process may include using tris(2-carboxyethyl) phosphine (TCEP) or dithiothreitol (DTT) to reduce the dissolved keratin again which can gain thiol functional groups. This process may be performed during the sodium sulfide treatment and cleanup the thiols that reacted with each other and formed disulfide bonds. These disulfide bonds may not be reactive with substances such as malcimide or vinyl sulfone.

Once dissolved, the kerateins are stable in solution without the denaturing agent for finite periods. Therefore, the denaturing agent can be removed without the resultant precipitation of kerateins. Regardless of the fractionation/purification process used, the resulting kerateins can be concentrated and lyophilized.

In some examples, keratin proteins can be freeze-dried (lyophilized) to achieve storage conditions while maintaining protein stability. In some examples, lyophilization is used to produce a protein cake of purified protein, and to stabilize the extracted keratin proteins. Methods known in the art such as shell freezing followed by vacuum or bulk freezing and applying high heat tend to degrade proteins. In some examples, a keratin protein cake, including keratein alpha or gamma is produced by a lyophilization of a clarified keratin protein solution, optionally after dialysis.

In some examples, the clarified protein solution post-dialysis is bulk frozen at about −40° C., and then a vacuum is applied until the containment containing the solution reaches about 250 torr. In some examples, heat is then applied in a step-wise fashion, bringing the material to about 0° C., then to about 25° C., then to about 37° C., while maintaining 250 torr pressure. In some examples, the lyophilization process occurs over a 24 hour period.

In some examples, precise grinding of the lyophilized material aids in the homogeneity of reconstitution and protein stability. Previous methods involve crude grinding methods, including grinding or chopping of the material in a laboratory blender. In the present invention, some examples employ a commercial grinding apparatus to machine the material to a homogenous particle size. In some examples, a pharmaceutical mill is employed, and in some examples the resulting particle size is about 1000 microns or less in diameter.

In some examples, the static charge from the ground material may optionally be removed, and in some cases the ground material is deionized.

Hydrogels, which are three-dimensional networks capable of absorbing copious amounts of water, can be prepared by weighing the appropriate keratin lyophilized powder or powders into an aqueous solution. In some examples, to form a hydrogel, the keratein powders were diluted with, for example, sterile phosphate buffered saline (PBS), sterile water, and/or saline, to generate a desired percent mass to volume ratio. Before forming the hydrogels or using any materials in vivo, the solutions used may be sterilized. In one example, the solutions can be sterilized by filtering the aqueous solution through a membrane that has a pore size sufficient to remove contaminants, such as a 0.22 micron pore size. In some examples, which are presented herein merely as an example, the hydrogel may include about 1% to about 99% or more by weight keratein, or about 5% to about 80%, or about 50% to about 80%. The keratein may be alpha-keratein or gamma-keratein, or some combination thereof. In some examples, the keratein in the hydrogel may include about 0.5 wt % to about 50 wt %, or about 1 wt % to about 30 wt %, of alpha-keratein or gamma-keratein, and mixtures thereof.

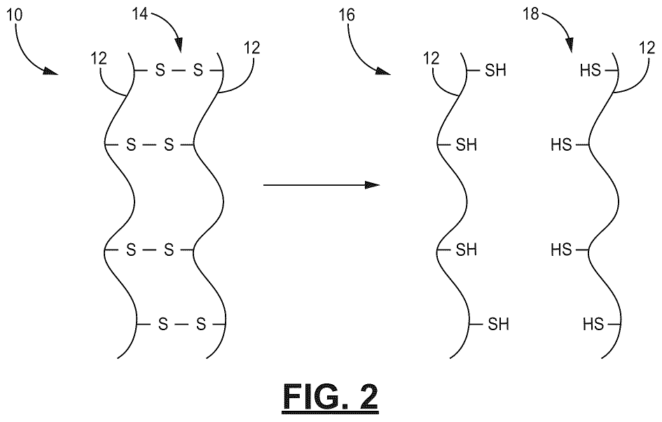

As shown schematically in FIG. 2, following the reduction reaction of FIG. 1, the keratein 10 includes elongate protein chains 12 linked by disulfide (S—S) bonds 14. Following additional reduction, dialysis and lyophilization, and incorporation into sterile PBS or water to form a hydrogel, a water-soluble keratein 16 is formed that includes thiol (S—H) groups 18 along the length of the elongate protein chains 12.

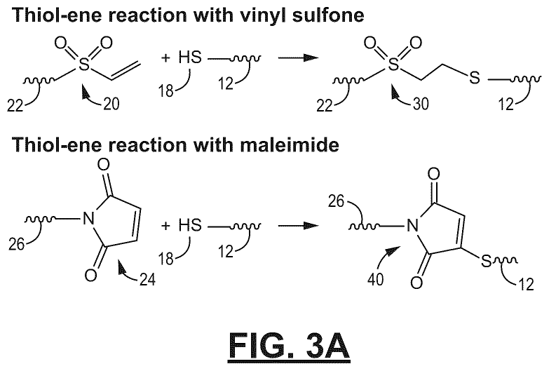

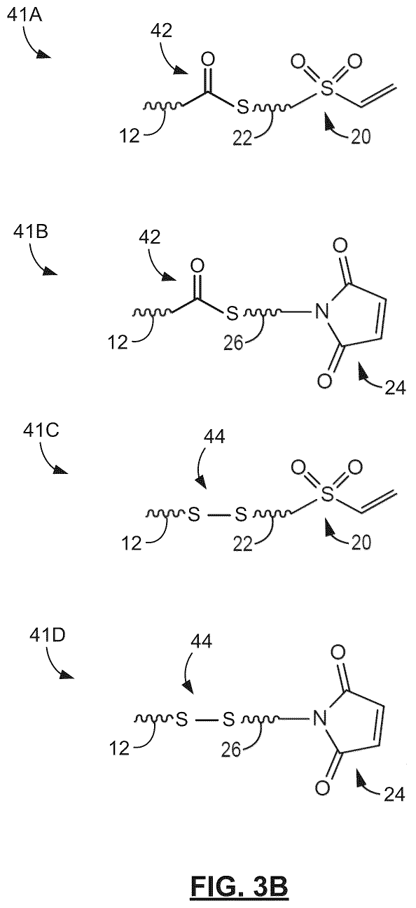

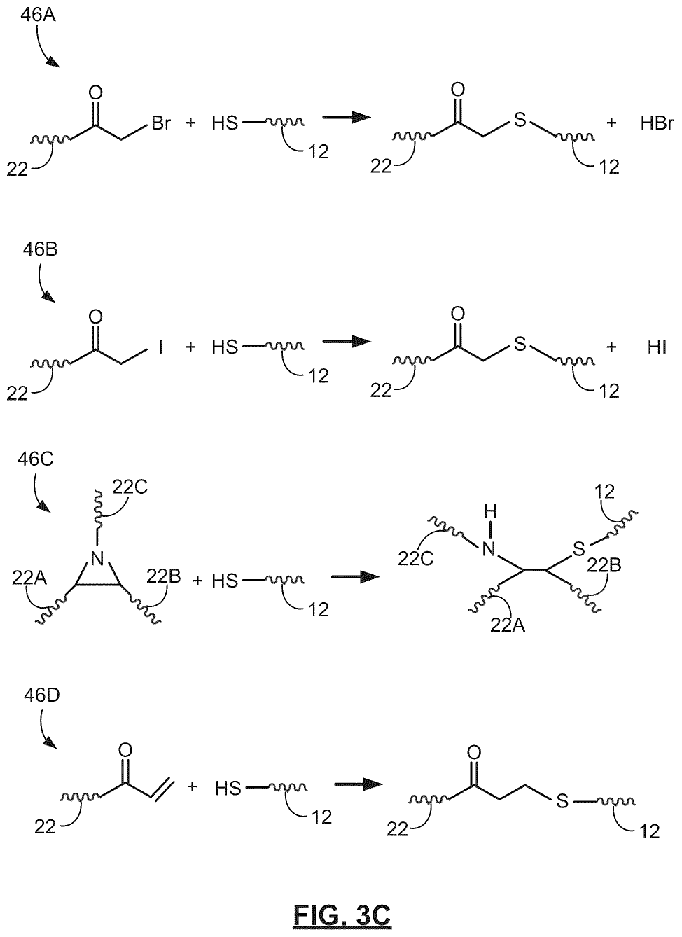

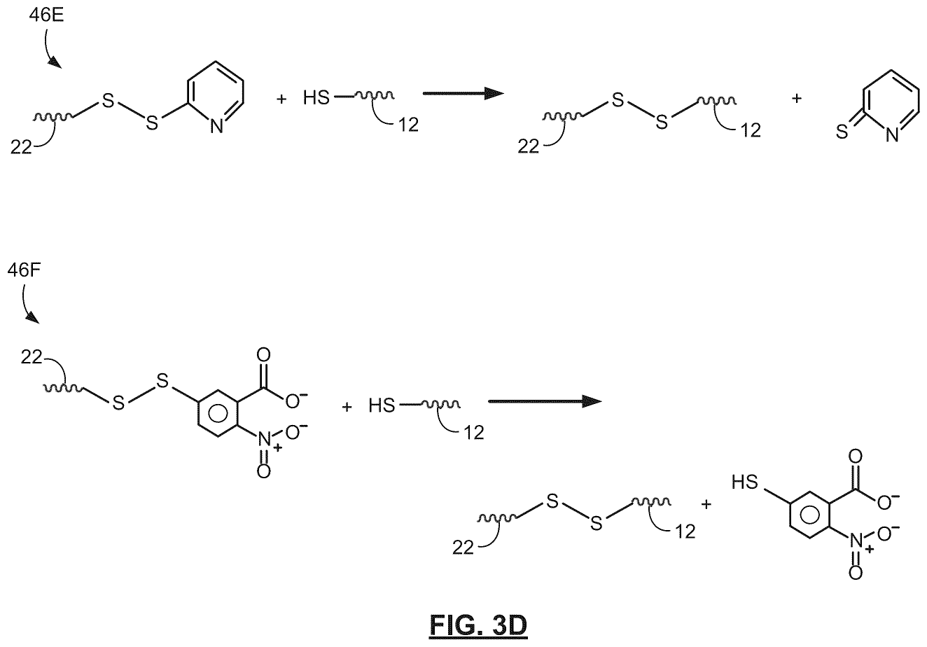

Referring now to the schematic representation in FIG. 3A, in one example the thiol groups 18 on the elongate keratein protein chains 12 (FIG. 2) are reacted with a vinyl sulfone compound 20 that optionally includes at least one water soluble polymer or copolymer 22. In this application, the term polymer or copolymer includes monomers, oligomers, polymers, copolymers, and mixtures and combinations thereof. The reaction, which produces a thioether sulfone linking group 30 that crosslinks the protein chains 12, does not require a catalyst, may be conducted by stirring in a preferably slightly alkaline aqueous solution or in organic solvent, and in some examples can lead to full conversion in less than about 24 hours. Polymers with pendant vinyl sulfone groups can be directly prepared for example by ring-opening polymerization resulting in polymers that can be subsequently reacted with the thiol groups on the keratein proteins.

In another example, the thiol groups 18 on the elongate protein chains 12 (FIG. 2) can be reacted with a maleimide compound 24 that may optionally include at least one water soluble polymer or copolymer 26. The reaction between the thiols and the maleimide, which is a Michael addition reaction, generates a thiosuccinimide linking group 40 that crosslinks the protein chains 12. The nucleophilic addition of thiols to maleimide does not require any heat or catalyst, and simple stirring of the two reactants at room temperature and a pH of about 2 to about 8.5 or about 6.5 to about 7.5, is often sufficient to achieve complete conversion. In some examples, the reaction between the thiols and the maleimide is referred to as click chemistry, as the reaction occurs under simple reaction conditions, uses readily available starting materials and reagents, requires no organic solvents, and provides an easily isolatable product.

The water soluble polymer or copolymer 22, 26 used in the formation of the respective thioether sulfone linking groups 30 and the thiosuccinimide linking groups 40 may vary widely depending on the intended application. In various examples, which are not intended to be limiting, the linking groups 30, 40 are water soluble polymers or copolymers with at least two maleimide or vinylsulfone moieties, and the maleimide or vinylsulfone moieties may optionally be functionalized. In various examples, the linking groups 30, 40 can be a linear polymer with at least two maleimide or vinylsulfone functional groups; or a linear or branched polymer containing at least two pendant maleimide or vinylsulfone functional groups (for example, modified from poly(allyl glycidyl ether)-b-poly(ethylene oxide)-b-poly(allyl glycidyl ether)); or a multi-arm polymer (for example, a 4-arm PEG with maleimide functional groups and a pentaerythritol core); or a dendric polymer.

In some examples, the water soluble polymer or copolymer with at least two maleimide or vinylsulfone functional groups forming the linking groups 30, 40 can include monomeric or copolymeric units such as poly (ethylene oxide) (PEO), poly (vinyl alcohol) (PVA), poly(acrylic acid) (PAA), polyacrylamide (PAA), poly(N-(2-hydroxypropyl) methacrylamide) (PHPMA), poly (ethylene glycol) (PEG), a polysaccharide, or other water soluble polymers and their copolymers. In another example, the linking groups 30, 40 can be a biopolymer modified with maleimide or vinylsulfone functional groups such as, for example, a polynucleotide (a DNA or RNA molecule), a polypeptide (a protein), and combinations thereof.

In some examples, the linking groups 30, 40 are multifunctional, which in this application means that a plurality of functional groups 22, 26 may be present on each linking group. For example, each linking group 30, 40 may include 2, 3, 4, or more functional groups. Although thioether sulfone linking group 30 and thiosuccinimide linking group 40 are two example linking groups described herein, other linking groups may be formed using other sulfhydryl-reactive chemical groups that can be reacted with thiols. As shown in FIGS. 3A, 3C, and 3D, example sulfhydryl-reactive chemical groups that may be used for linking groups described herein may thus include haloacetyls, maleimides, aziridines, acryloyls, arylating agents, vinylsulfones, pyridyl disulfides, TNB-tbiols, and disulfide reducing agents.

In some examples, the multifunctional crosslinkers have a molecular weight of about 100 Da to about 100 kDa, or about 10 kDa to about 50 kDa.

In various examples, which are not intended to be limiting, the multifunctional crosslinkers can have a branched structure with at least 2 arms having a malcimide or vinylsulfone functional group thereon, or at least 4 arms, or at least 8 arms. In some examples, the multifunctional crosslinkers can be highly branched molecules such as dendrimers. In one example the dendrimers can have a circular or spherical shape with a core and arms extending outwardly from the core, wherein the arms have thereon a selected functional group. In this manner, multiple linking groups can create multifunctional crosslinkers forming a network that makes up example hydrogels described herein.

In some examples, multiple PEG (or any of the other water soluble polymers or copolymers above) arms can be incorporated into the linking groups 30, 40 to enhance or modify the properties of the crosslinked hydrogel. By incorporation of such multifunctional modifiers into the crosslinks between the protein chains, the properties of the biomaterial, such as hydrophilicity, porosity, swelling, degradation, mechanical properties, loading capacity for a pharmacologically-active agent, release kinetics of a pharmacologically-active agent, etc. may be adjusted through tuning of the physical and chemical properties of the multifunctional modifier. For example, multifunctional PEG modifiers may improve hydrophilicity and solubility, which will further influence swelling, degradation, and mechanical properties of the crosslinked hydrogel, while linear PEG modifiers may serve as hydrophilic brushes that may reduce the potential electrostatic interactions and thus facilitate the release of hydrophilic solutes. In one example, multifunctional modifiers including a PEG-poly(lactic acid)-PEG segment may impart the ability to degrade (e.g., in vivo) the crosslinked hydrogel, and may also vary the hydrophilicity/hydrophobicity of the hydrogel. In some examples, the molecular weights the PEG modifiers may be tuned to adjust the degree of exposure of multifunctional ligands and/or probes at the surface of the crosslinked hydrogel.

In another example, as shown in the example of FIG. 3B, thioester or disulfide containing PEG modifiers may be used to allow for rapid dissolution of the hydrogel. FIG. 3B is a schematic representation of example rapidly dissolvable biomaterials, such as hydrogels, including keratin. The rapidly dissolvable biomaterials may be formed using similar reactions with thiols as described in FIG. 3A, but result in different crosslinkers for the keratin chains. In the examples, of FIG. 3B, the hydrogels 41A, 41B, 41C, and 41D (hydrogels 41) include a functional group, such as thioester or disulfide, that are configured to enable rapid dissolution of the respective hydrogel. In some examples, dissolution can occur from the use of a thiol-containing molecule such as glutathione or cysteine. Adding the thiol-containing small molecule solution can cleave the thioester or disulfide bonds and result in rapid dissolution of the hydrogel. Rapid dissolution of the hydrogel may occur in less than 10 minutes, less than 5 minutes, or less than 3 minutes in some examples, depending on the hydrogel or small molecule used for dissolution.

Hydrogels 41 can include keratin protein chains 12 and one or more of functional groups 22, 26. Example hydrogel 41A includes vinyl sulfone compound 20 that includes at least one water soluble polymer or copolymer 22 and thioester 42 on keratin protein chain 12. Example hydrogel 41B includes maleimide compound 24 that includes at least one water soluble polymer or copolymer 26 and thioester 42 on keratin protein chain 12. Example hydrogel 41C includes vinyl sulfone compound 20 that includes at least one water soluble polymer or copolymer 22 and disulfide 44 on keratin protein chain 12. Example hydrogel 41D includes maleimide compound 24 that includes at least one water soluble polymer or copolymer 26 and disulfide 44 on keratin protein chain 12.

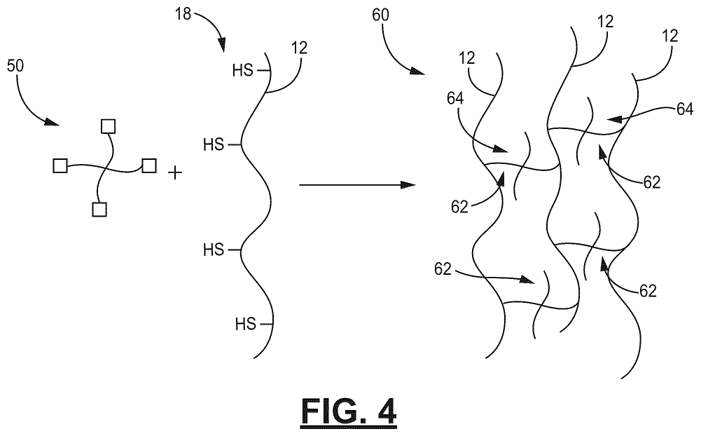

Referring to the schematic illustration in FIG. 4, an aqueous solution including a “4-arm” multifunctional PEG crosslinker 50 with maleimide functional groups can be mixed with an aqueous pre-gel solution including water soluble keratein with elongate protein chains 12 and thiol groups 18 to form a crosslinked hydrogel 60. The crosslinked hydrogel 60 includes elongate keratein protein chains 12 bound by thiosuccinimide crosslinks 62 extending generally normal to the longitudinal direction of the protein chains. The crosslinks 62 include multiple free PEG arms 64, which can be used to control the properties of the crosslinked hydrogel 60 including, for example, at least one of hydrophilicity, porosity, swelling, degradation, mechanical properties, loading capacity for a pharmacologically-active agent, release kinetics of a pharmacologically-active agent, and the like. In some examples, unreacted thiol functional groups in the crosslinked hydrogels can be used to modify the properties of the hydrogels.

When a first aqueous pre-gel solution including the hydrogel and a second aqueous solution including the crosslinking compound are mixed at room temperature of about 20° C. to about 37° C., rapid in-situ gelation occurs in less than about 100 seconds, less than about 50 seconds, less than about 10 seconds, less than about 5 seconds, less than about 2 seconds, or less than about 1 second. No catalysts or organic solvents are needed to move the reaction to completion, and substantially no by-products are produced as the reaction proceeds. In some examples, the reaction can proceed more quickly at a higher pH level.

Gelation time and gel properties for the crosslinked hydrogel can be tuned by, for example, adjusting the pre-gel solution concentration of keratein, crosslinking agent molecular weight, pH, and degree of crosslinker functionality. In some examples, the gelation time is generally dependent on the keratin protein and the crosslinking compound concentration in the pre-gel solution, as higher pre-gel concentration leads to faster gelation. In contrast, lower concentration of the crosslinking compound, or multiple functional groups on the crosslinking compound, can lead to slower gelation times. In general, the gelation time is dependent on the molar concentration of the functional groups. When using the same compounds (same molecular weight and number of functionality), the higher polymer/biopolymer concentration leads to faster gelation. In some examples, if the molecular weight of the crosslinking compound is increased, but the mass concentration of the crosslinking compound stays approximately the same, the relative molar concentration of the functional groups is decreased, therefore the gelation would be expected to be slower.

In one example, using a multifunctional crosslinker with 4 PEG arms and maleimide functional groups and having a molecular weight of about 10 k Daltons, gelation to form a hydrogel occurred in less than 1 second at a 10 wt % concentration of keratein and 10 wt % 4-arm PEG in the pre-gel solution. The result may be a hydrogel that has 10 wt % of polymer/biopolymer. In another example, gelation at 5 wt % keratein concentration in the pre-gel solution and 10 kDa 4-arm-PEG maleimide at 5 wt % occurred at 1.5 seconds, while gelation time increased to 26.5 seconds for 40 kDa 4-arm-PEG maleimide crosslinker, respectively.

The crosslinked hydrogel product can also have relatively low viscosity, which can be used to provide an injectable composition for use as an embolic agent. In one example, which is not intended to be limiting, a pre-gel solution with 8 wt % keratein protein was reacted in a tube with multifunctional 4-arm-PEG maleimide crosslinker having a molecular weight of about 40 kDa to form a crosslinked hydrogel. The crosslinked hydrogel could be easily drawn from the tube into a syringe and then pushed through a 2.4 French (Fr) (0.8 mm) catheter before the crosslinking reaction was completed. The crosslinking time of about 20-30 seconds made it possible to push the partially crosslinked hydrogel through the catheter into a vessel. Once the hydrogel is fully crosslinked, the material forms a substantially solidified embolic agent in the vessel.

The crosslinked hydrogel has very good mechanical properties. In some examples, the modulus of the crosslinked hydrogel is also dependent on the pre-gel concentration as well as the molecular weight of the multifunctional PEG maleimide crosslinker. In some examples, which are not intended to be limiting, the crosslinked gels have G′ values in the 10³ to 10⁴ Pa range.

The shape of the hydrogel is generally dependent on the shape of the void in which the pre-gel cursors are mixed. In some examples, which are provided as an example, the reaction materials form a liquid embolic that will flow into a designated space that needs occlusion, and form in that space a gel that has the shape of the blood vessel or aneurysm.

In some examples, the crosslinked hydrogel formed in the blood vessel or aneurysm can be a relatively permanent embolic material. In other examples, the selected crosslinker can provide the crosslinked hydrogel with desired degradation properties. In one example, which is not intended to be limiting, a PEG crosslinker can have an amide bond between the maleimide and an adjacent PEG repeat unit. If the amide bond is replaced with an ester linkage, the hydrogel can be degraded within a few hours to several days through ester hydrolysis. If the PEG crosslinker contains thiolester or disulfide bonds, as shown in the example hydrogels 41 of FIG. 3B, the hydrogel can be degraded or dissolved rapidly (e.g., within a time period between 1 to 5 minutes) by introducing a thiol-containing small molecule such as cysteine or glutathione as two examples.

If the hydrogel is a rapidly dissolvable biomaterial, the hydrogel may be slightly different in structure than the “4-arm” multifunctional PEG crosslinker 50 and crosslinked hydrogel 60. For example, if PEG crosslinker 50 includes maleimide functional groups represented by the boxes at the end of each arm of PEG crosslinker 50, thioester or disulfide may be located between the maleimide functional group and the respective arm of each arm in the PEG crosslinker 50. In some examples, the thioester, disulfide, or other group configured to enable rapid dissolving of the hydrogel may be located on each art of PEG crosslinker 50, but in other examples not every arm of PEG crosslinker 50 may include the example thioester or disulfide group. In any event, the thioester or disulfide, for example, may be located within some or all of the thiosuccinimide crosslinks 62 within the modified and rapidly dissolvable version of crosslinked hydrogel 60.

FIGS. 3C and 3D are a schematic representations of example reactions in which the thiol groups on the keratein of FIG. 2 is reacted with functional groups from functionalized crosslinking compounds to form covalent bonds. As discussed here, example sulfhydryl-reactive chemical groups that may be used for linking groups described herein may thus include baloacetyls, maleimides, aziridines, acryloyls, arylating agents, vinylsulfones, pyridyl disulfides, TNB-thiols, and disulfide reducing agents.

As shown in FIG. 3C, reaction 46A includes reacting thiol groups on the elongate protein chains 12 with a haloacetyl compound containing Bromine that may optionally include at least one water soluble polymer or copolymer 22. The reaction between the thiols and the haloacetyl compound generates a thioether linking group that crosslinks the protein chains 12. The nucleophilic addition of thiols to the haloacetyl compound can occur with simple stirring of the two reactants at room temperature and a pH of about 7.5 to about 9.0 to achieve complete conversion. Reaction 46B includes reacting thiol groups on the elongate protein chains 12 with a haloacetyl compound containing Iodide that may optionally include at least one water soluble polymer or copolymer 22. The reaction between the thiols and the haloacetyl compound generates a thioether linking group that crosslinks the protein chains 12. The nucleophilic addition of thiols to the haloacetyl compound can occur with simple stirring of the two reactants at room temperature and a pH of about 7.5 to about 9.0 to achieve complete conversion.

Also shown in FIG. 3C is reaction 46C which includes reacting thiol groups on the elongate protein chains 12 with a aziridine that may optionally include water soluble polymer or copolymers 22A, 22B, and 22C. The reaction between the thiols and aziridine can be performed in the presence of a catalyst such as DBU (1,8-Diazabicyclo[5.4.0]undec-7-ene) or ZnCl₂. Reaction 46D includes reacting thiol groups on the elongate protein chains 12 with an acryloyl compound that may optionally include water soluble polymer or copolymer 22. The reaction between the thiols and acryloyl generates a thioether linking group that crosslinks the protein chains 12.

As shown in FIG. 3D, reaction 46E includes reacting thiol groups on the elongate protein chains 12 with pyridyl disulfide that may optionally include at least one water soluble polymer or copolymer 22. The reaction between the thiols and pyridyl disulfide generates a disulfide bond linking group that crosslinks the protein chains 12 and generates a byproduct of pyridine-2-thione. Reaction 46F includes reacting thiol groups on the elongate protein chains 12 with TNB-thiols that may optionally include at least one water soluble polymer or copolymer 22. The reaction between the thiols and TNB-thiols generates a disulfide bond linking group that crosslinks the protein chains 12 and generates a byproduct of 5-thio-2-nitrobenzoic acid.

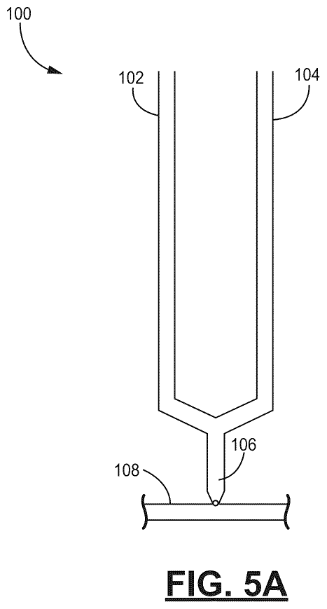

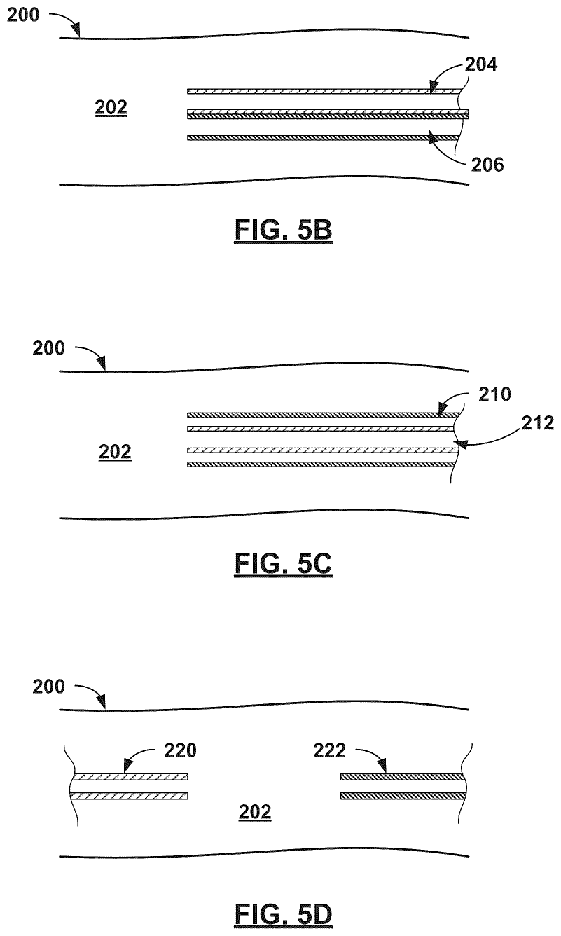

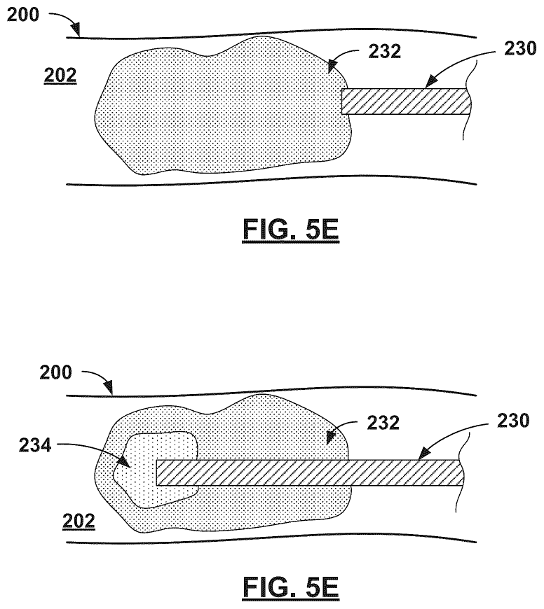

In some examples, if the crosslinked hydrogels may be configured for use as an embolic agent, a first aqueous pre-gel solution including the keratein proteins and a second aqueous solution including the multifunctional PEG maleimide crosslinker can be injected into an occludable vessel using an injection system 100 shown schematically in FIG. 5A. The injection system 100 includes a first catheter 102 and a second catheter 104, which are joined downstream at a nozzle 106. If the pre-gel solution is injected or drawn into the first catheter 102, and then the aqueous solution of the multifunctional PEG maleimide crosslinker can be injected or drawn into the second catheter 104. A crosslinked hydrogel is rapidly formed just downstream of the nozzle 106 in a vessel 108 to be occluded. In some examples, the length of the nozzle 106 is dependent on the hydrogel gelation time, and for faster gelation times, a shorter nozzle 106 may be used.

In some examples, gelation to form the hydrogel occurs extremely quickly, and the nozzle 106 is not required. In such examples a coaxial dual lumen system can be used, and no mixing of the reactants occurs prior to entry of the reactants into the blood vessel or aneurysm to be occluded. In some examples, the reactants may be reacted and delivered to the occlusion site in a single catheter.

In the two microcatheter arrangement, for example, the two solutions can be injected separately through each lumen. In the one lumen catheter arrangement, for example, the first solution including the keratein proteins will be injected. Then a small amount of saline will be injected, and the second solution including the crosslinkers can be injected after the saline. The small amount of saline serves as a spacer in the catheter to prevent mixing of the two polymer solutions inside the catheter.

FIGS. 5B, 5C, and 5D are conceptual diagrams of example catheters configured to deliver different materials to form embolic agents to occlude a blood vessel. The examples of FIGS. 5B, 5C, and 5D may be similar to system 100 of FIG. 5A. However, the catheters have different configurations for delivering the components of an embolic agent, such as a pre-gel solution and an aqueous solution or any other components described herein. The catheters may be directed through the vasculature and to the desired location within blood vessel 200 to which the embolic agents can be delivered. In examples in which there may be three components or more, each component may be delivered by a separate and respective catheter or lumen within a common delivery structure.

In the example of FIG. 5B, cross-sectional views of catheters 204 and 206 are provided such that catheters 204 and 206 are disposed in a side-by-side configuration. Catheters 204 and 206 may be disposed within lumen 202 of blood vessel 200. Catheters 204 and 206 may be separate from each other or attached at one or more locations, or even attached along their entire length. The distal openings of catheters 204 and 206 may be configured to be disposed with their distal openings aligned with each other or such that the distal opening of catheter 204 is disposed more distal of the distal opening of catheter 206, or vice versa.

In the example of FIG. 5C, cross-sectional views of catheters 210 and 212 are provided such that catheters 210 and 212 are disposed in a concentric configuration such that catheter 212 is disposed within the lumen of catheter 210. In this configuration, the wall of catheter 212 separates the lumen of catheter 212 from the lumen of catheter 210. Catheters 210 and 212 may be disposed within lumen 202 of blood vessel 200. Catheters 210 and 212 may be separate from each other or attached at one or more locations. The distal openings of catheters 210 and 212 may be configured to be disposed with their distal openings aligned with each other or such that the distal opening of catheter 212 is disposed more distal of the distal opening of catheter 210, or vice versa.

In the example of FIG. 5D, cross-sectional views of catheters 220 and 222 are provided such that catheters 220 and 222 can be disposed within vessel 200 in an opposing configuration such that the distal openings of each catheter 220 and 222 are pointed at each other to deliver the components of the embolic agents toward each other.

FIGS. 5E and 5F are conceptual diagrams illustrating a method for delivering different components to form an embolic agent similar to the system of FIG. 5A. However, instead of two catheters, a single catheter 230 may be positioned within blood vessel 200 such that the distal end of catheter 230 is disposed at a target location within lumen 202. Once positioned, as shown in FIG. 5E, first solution 232 can be injected from the distal end of catheter 230 and into lumen 202 of vessel 200. In some examples, first solution 232 will partially fill lumen 202, and in other examples, first solution 232 will fill the cross-sectional area of lumen 202.

After first solution 232 is injected, the user or system may move catheter 230 distally, or further into, first solution 232 as shown in FIG. 5F. Once within the bolus of first solution 232, the user or system may inject a small amount of a non-reactive fluid, such as saline, to act as a spacer or transition fluid between first solution 232 and second solution 234, for example, within catheter 230. An example volume of saline may be 100 micro liters of saline. In some examples, the saline may not completely fill catheter 230, but in other examples, the saline may be dispensed from the distal end of catheter 230 before second solution 234 is next added to catheter 230. In other examples, first solution 232 and second solution 234 may not need to be separated within catheter 230 (either being delivered serially, one after the other, or delivered together such that at least some mixing occurs within catheter 230). After saline is injected into catheter 230, the user or system may inject second solution 234 through catheter 230 and into the bolus of first solution 232. In this manner, first solution 232 and second solution 234 may react to form the embolic agent within blood vessel 200. In one example, first solution 232 may be a first aqueous pre-gel solution including the keratein proteins and second solution 234 may be a second aqueous solution including the multifunctional PEG maleimide crosslinker. When combined, the embolic hydrogel may be formed to occlude vessel 200. As described herein, first and second solutions 232 and 234 may include other components that can be combined to form the occlusion.

In some examples, the first and second catheters, the nozzle, and the first and second aqueous solutions can be provided to a practitioner in the form of a kit. In one example, the kit includes a powdered water soluble keratein, which can be provided in a nitrogen-charged container capped with a rubber septum. A second container can include a powdered crosslinking material including a polymeric compound with at least two maleimide or vinylsufone functional groups. The powdered keratein and crosslinker can be used to form a first aqueous solution and a second aqueous solution to be mixed together. The kit can optionally include an arrangement of one or more catheters of a suitable diameter and length for treatment of a patient.

The kit can optionally include ancillary items including, but not limited to, instructions, antiseptic, adhesive tapes, gloves, and the like. In some examples, the kit can optionally include additional occlusion devices such as, for example, a balloon catheter for temporary vessel occlusion. The kit may also include a device configured to accept two syringes such that the device enables simultaneous injection of the two solutions from the two syringes using only one hand.

The crosslinked hydrogels of the present disclosure can be used in a wide variety of applications including, but not limited to, wound dressing materials, diapers, catamenial devices, drug delivery devices, implants, biosensors, contact lenses, tissue scaffolds, cell transplantation matrices, embolic agents, three-dimensional (3D) bioprinting, and the like.

In some examples, the crosslinked hydrogel can be loaded with a pharmaceutically active agent such as, for example, a drug. The degradation of the hydrogel can be adjusted to provide a resorbable or permanent embolic agent in the form of microparticles or liquid. In various examples, the pharmacologically-active agent may be entrained within the crosslinked hydrogel, or it may be covalently attached to the keratein protein or the multifunctional crosslinker.

In various examples, the pharmacologically-active material may include vulnerary agents, hemostatic agents, antibiotics, anthelmintics, antifungal agents, hormones, anti-inflammatory agents, proteins, polypeptides, oligonucleotides, cytokines, enzymes, and the like. In a further example, the crosslinked hydrogels may be used to administer a pharmacologically-active agent to a patient by, for example, by packing into a surgical or traumatic wound.

Likewise, the crosslinked hydrogels may be useful as scaffolds to support living cells. The crosslinked hydrogels can be used as biomechanical devices to support living cells within the bulk of the material, providing a three-dimensional support network in which the cells can grow and proliferate. Such cells may be entrained within the crosslinked hydrogel. In one example example, crosslinked hydrogels that contain cells can be implanted into a patient in need of such cells, and can provide a support structure for monocytes, fibroblasts, keratinocytes, chondrocytes, myoblasts, endothelial progenitor cells, stem cells, and the like.

The crosslinked hydrogels of the present disclosure will now be further described in the following non-limiting examples.

### EXAMPLES

### Example 1

### Materials & Methods

### Keratin Extraction and Characterization

Human hair was washed with detergent to remove contamination, and de-lipidized with chloroform and methanol. The hair was air dried, cut into smaller pieces and then immersed in 0.5 M sodium sulfide solution (pH 10) at 40° C. overnight. The resulting suspension was centrifuged, while the superatant was dialyzed against deionized water for three days and then lyophilized to produce a brown powder. The keratin was further treated with tris(2-carboxyethyl) phosphine hydrochloride (TCEP), dialyzed and lyophilized to produce keratein. The keratein had a thiol content of about 1 mmol/gram.

### Crosslinked Hydrogel Preparation

Crosslinked hydrogel formation was accomplished by mixing separate solutions of multi-functionalized PEG-maleimide and the high thiol content keratein, prepared individually in phosphate buffered saline (PBS) at various polymer concentrations.

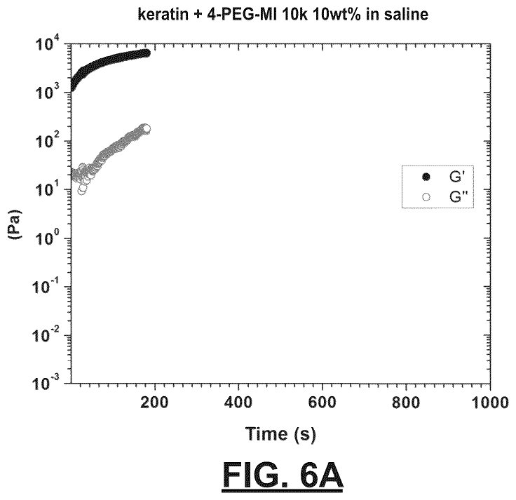

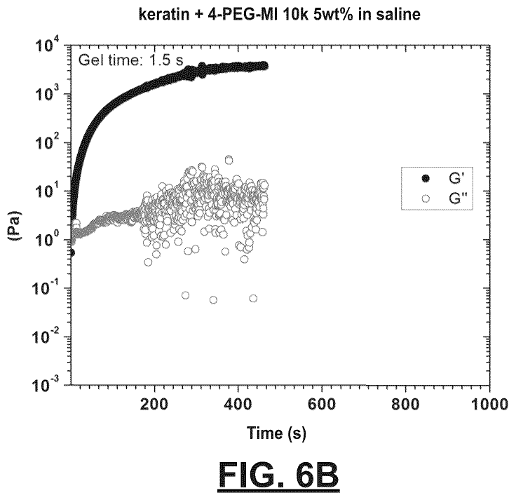

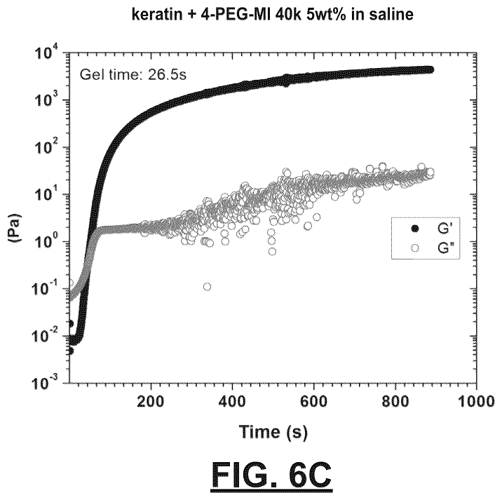

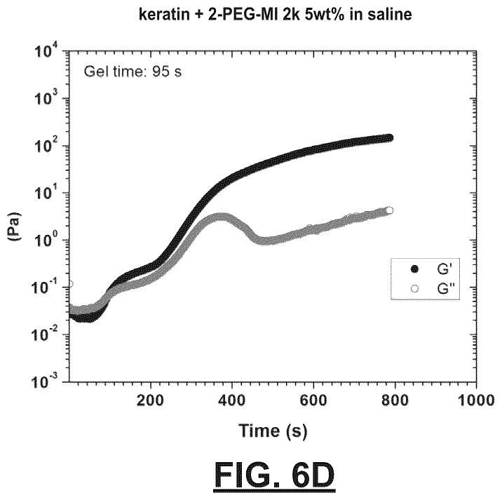

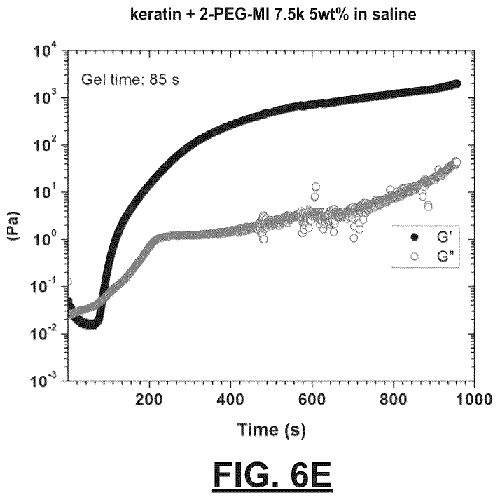

A pre-gelation solution of 10 wt % keratein in saline was mixed with a multifunctional PEG maleimide crosslinker 10 wt % solution in saline with a molecular weight of 10 kDa and having 4 PEG arms end-capped with maleimide functional groups. As shown in the plot of FIG. 6A, the crosslinked hydrogel formed in a gel time of about −0.5 seconds.

A pre-gelation solution of 5 wt % keratein in saline was mixed with a multifunctional PEG maleimide crosslinker 5 wt % solution in saline with a molecular weight of 10 kDa and having 4 PEG arms end-capped with maleimide functional groups. As shown in the plot of FIG. 6B, the crosslinked hydrogel formed in about 1.5 seconds.

A pre-gelation solution of 5 wt % keratein in saline was mixed with a multifunctional PEG maleimide crosslinker 5 wt % solution in saline with a molecular weight of 40 kDa and having 4 PEG arms end-capped with maleimide functional groups. As shown in FIG. 6C, the crosslinked hydrogel formed in about 26.5 seconds.

A pre-gelation solution of 5 wt % keratein in saline was mixed with a multifunctional PEG maleimide crosslinker 5 wt % solution in saline with a molecular weight of 2 kDa and having 2 PEG arms end-capped with maleimide functional groups. As shown in FIG. 6D, the crosslinked hydrogel formed in about 95 seconds.

A pre-gelation solution of 5 wt % keratein in saline was mixed with a multifunctional PEG maleimide crosslinker 5wt % solution in saline with a molecular weight of 7.5 kDa and having 2 PEG arms end-capped with maleimide functional groups. As shown in FIG. 6E, the crosslinked hydrogel formed in about 85 seconds.

The results of FIGS. 6A-6E indicate that the gelation time and gel properties were adjustable through changing the number of reaction sites of PEG, the molecular weight of PEG, and the concentration of the polymer solution.

### Example 2

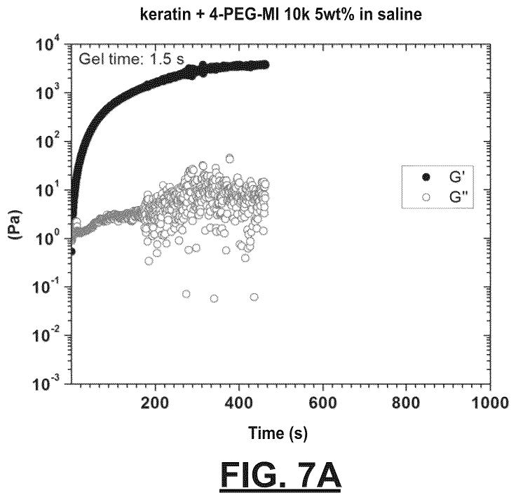

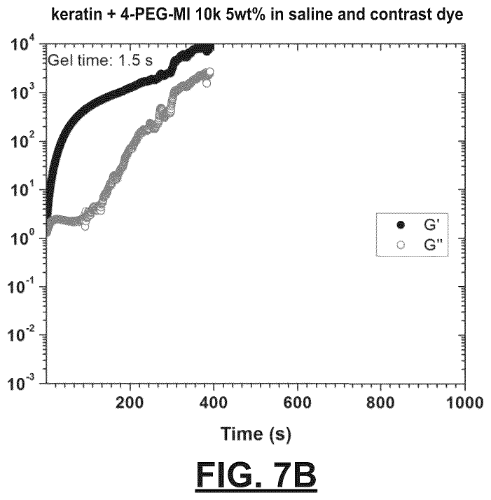

A pre-gelation solution of 5 wt % keratein in saline was mixed with a multifunctional PEG maleimide crosslinker 5 wt % solution in saline with a molecular weight of 10 kDa and having 4 PEG functional groups. As shown in the plot of FIG. 7A, the crosslinked hydrogel formed in a gel time of about 1.5 seconds.

Next, a contrast dye was added to the pre-gelation solution described in FIG. 7A. As shown in FIG. 7B, the gel time remained at 1.5 seconds, which indicated that the addition of the contrast dye did not have an impact on the gelation time of the crosslinked hydrogel.

### Example 3

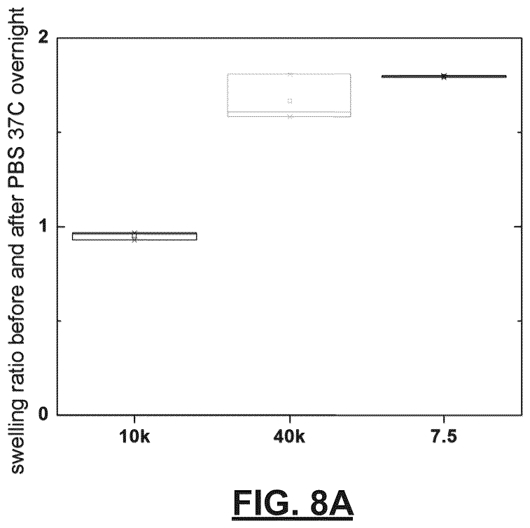

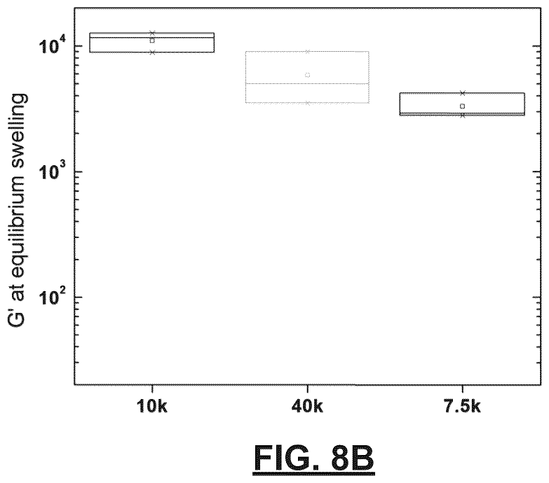

A pre-gelation solution of 5 wt % keratein in saline was mixed with three different multifunctional PEG maleimide crosslinker at 5 wt % with a molecular weights of 10 kDa, 40 kDa and 7.5 kDa. The 10 kDa and 40 kDa crosslinkers had 4 maleimide functional groups, while the 7.5 kDa crosslinker had only 2 maleimide functional groups. As shown in the plot of FIG. 8A, the crosslinked hydrogel formed from each of these crosslinkers exhibited different levels of swelling when stored overnight in PBS at 37° C. The crosslinked hydrogel formed from the 40 kDa crosslinkers (4 armed PEG with maleimide functional groups), and the 7.5 kDa crosslinker (2 armed PEG with maleimide functional groups) exhibited the most swelling, while the crosslinked hydrogel from the 10 kDa crosslinker (4 armed PEG end capped with maleimide functional groups) exhibited almost no significant swelling. While not wishing to be bound by any theory, these results indicate that the molecular weight of PEG arm has an impact on the swelling of the hydrogel upon storage, with higher molecular weight PEG arms providing more hydrogel swelling, possibly due to the relaxation of the polymer chains during overnight storage. The fast gelation time of the hydrogels with more functional groups crosslinked some of the polymer chains at their entangled state and the some of the entanglement was relieved when the hydrogel was placed in saline overnight.

FIG. 8B shows the G′ values at equilibrium swelling for the crosslinked hydrogels of this example, and indicates that the crosslinked hydrogel formed from the 10 kDa crosslinker (4 PEG functional groups) had the highest G′ value, while the crosslinked hydrogel formed from the 40 kDa crosslinker (4 PEG functional groups) had a somewhat lower G′, and the crosslinked hydrogel formed from the 7.5 kDa crosslinker (2 PEG functional groups) bad the lowest G′ value.

The following examples are described herein.

Example 1. A biomaterial comprising keratin proteins crosslinked with linking groups chosen from thiosuccinimide, thioether sulfone, and mixtures and combinations thereof.

Example 2. The biomaterial of example 1, wherein the biomaterial is in the form of a hydrogel.

Example 3. The biomaterial of example 2, wherein the linking groups comprise a linear or branched water soluble polymer or copolymer with at least two functional moieties chosen from maleimide, vinylsulfone, and combinations thereof.

Example 4. The biomaterial of example 3, wherein the water soluble polymer or copolymer comprises monomeric or copolymeric units chosen from poly(ethylene oxide) (PEO), poly(vinyl alcohol) (PVA), poly(acrylic acid) (PAA), polyacrylamide, poly(N-(2-hydroxypropyl) methacrylamide) (PHPMA), a polysaccharide, poly(ethylene glycol) (PEG), and combinations thereof.

Example 5. The biomaterial of example 4, wherein the water soluble polymer or copolymer comprises PEG.

Example 6. The biomaterial of example 3, wherein the water soluble polymer or copolymer comprises a multi-arm polymer.

Example 7. The biomaterial of example 3, wherein the water soluble polymer or copolymer comprises a biopolymer.

Example 8. The biomaterial of any of examples 1 through 7, wherein the linking groups comprise thiosuccinimides derived from a thiol-ene reaction between thiol groups on the keratin proteins and a maleimide compound comprising a water-soluble polymer or copolymer.

Example 9. The biomaterial of any of examples 1 through 8, wherein the linking groups comprise thioether sulfones derived from a thiol-ene reaction between thiol groups on the keratin proteins and a vinyl sulfone compound comprising a water-soluble polymer or copolymer.

Example 10. The biomaterial of any of examples 1 through 9, wherein the keratin proteins are obtained from human hair.

Example 11. The biomaterial of example 10, wherein the keratin proteins comprise keratein.

Example 12. The biomaterial of example 1, wherein the biomaterial comprises a hydrogel comprising the keratin proteins crosslinked with one or more of the thiosuccinimide linking groups.

Example 13. The biomaterial of example 1, wherein the biomaterial comprises a hydrogel comprising the keratin proteins crosslinked with one or more of the thioether sulfone linking groups.

Example 14. A method for making the biomaterial of any of examples 1 through 13, wherein the method comprises reacting a first aqueous pre-gel solution comprising a water soluble keratein with an aqueous solution comprising a crosslinking compound chosen from maleimide, vinyl sulfone, and mixtures and combinations thereof, to create the biomaterial comprising the keratin proteins crosslinked with at least one of the thiosuccinimide or the thioether sulfone.

Example 15. A system for delivering components that react to form the biomaterial of any of examples 1 through 13, wherein the system comprises at least one catheter configured to be disposed within a blood vessel, and wherein the at least one catheter is configured to deliver the components that react to form, in the blood vessel and outside of the at least one catheter, the keratin proteins crosslinked with at least one of the thiosuccinimide or the thioether sulfone.

Example 16. A hydrogel comprising a biomaterial in an aqueous medium, wherein the biomaterial comprises keratin proteins crosslinked with thiosuccinimide linking groups.

Example 17. The hydrogel of example 16, wherein the thiosuccinimide linking groups comprise: a water soluble polymer or copolymer with PEG monomeric units; and at least two functional groups chosen from maleimide, vinylsulfone, and combinations thereof.

Example 18. The hydrogel of example 17, wherein the keratin proteins comprise keratein.

Example 19. The hydrogel of example 18, wherein the keratin proteins consist essentially of keratein.

Example 20. The hydrogel of any of examples 16 through 19, wherein the thiosuccinimide linking groups are derived from a thiol-ene reaction between thiol groups on the keratein and a maleimide compound comprising a water soluble copolymer or copolymer.

Example 21. The hydrogel of example 20, wherein the water soluble polymer or copolymer comprises PEG monomeric units.

Example 22. The hydrogel of any of examples 16 through 21, wherein the hydrogel further comprises a therapeutic agent.

Example 23. The hydrogel of example 22, wherein the therapeutic agent comprises at least one drug.

Example 24. The hydrogel of example 22, wherein the therapeutic agent is entrained in the hydrogel.

Example 26. The hydrogel of any of examples 16 through 25, wherein the linking groups comprise multiple arms.

Example 27. The hydrogel of any of examples 16 through 26, wherein the linking groups comprise a biopolymer.

Example 28. A method for making a hydrogel, the method comprising reacting a first aqueous pre-gel solution comprising a water soluble keratein with a second aqueous solution comprising a crosslinking compound chosen from maleimide, vinyl sulfone, and mixtures and combinations thereof.

Example 29. The method of example 28, wherein the crosslinking compound comprises a water soluble polymer or copolymer.

Example 30. The method of example 29, wherein the water soluble polymer or copolymer comprises PEG monomeric units.

Example 31. The method of any of examples 28 through 30, further comprising introducing a therapeutic agent into the aqueous pre-gel solution.

Example 32. The method of any of examples 28 through 31, wherein reacting the first aqueous pre-gel solution comprising the water soluble keratin with the second aqueous solution comprises reacting the keratein with a maleimide functionalized PEG compound at a temperature of about 20° C. to about 37° C. for less than about 100 seconds.

Example 33. The method of any of examples 28 through 32, wherein the hydrogel comprises crosslinks with at least two moieties chosen from maleimide, vinylsulfone, and combinations thereof.

Example 34. The method of example 33, the crosslinks comprise a plurality of arms.

Example 35. A method for occluding a blood vessel, the method comprising introducing into the blood vessel an embolic agent comprising keratin proteins crosslinked with linking groups chosen from thiosuccinimide, thioether sulfone, and mixtures and combinations thereof.

Example 36. The method of example 35, wherein the embolic agent is in the form of a hydrogel.

Example 37. The method of any of examples 35 and 36, wherein the linking groups further comprise at least one water soluble polymer or copolymer.

Example 38. The method of example 37, wherein the water soluble polymer or copolymer comprises poly (ethylene glycol) (PEG) monomeric units.

Example 39. The method of any of examples 35 through 38, wherein the linking groups comprise thiosuccinimides derived from a thiol-ene reaction between thiol groups on the keratin proteins and a PEG compound with maleimide functional groups.

Example 40. The method of any of examples 35 through 39, wherein the keratin proteins are obtained from human hair.

Example 41. The method of example 40, wherein the keratin proteins comprise keratein.

Example 42. The method of any of examples 35 through 41, wherein the linking groups comprise a plurality of arms.

Example 43. A kit for conducting an embolization procedure, the kit comprising: a first powdered water soluble keratein; a crosslinker comprising at last two compounds of at least one of a maleimide or a vinylsulfone compound; and at least one catheter configured to deliver the aqueous solution, the keratein, and the crosslinker to an occlusion site to form a hydrogel at the occlusion site.

Various examples of the invention have been described. These and other examples are within the scope of the following claims.

1. A biomaterial comprising keratin proteins crosslinked with linking groups chosen from thiosuccinimide, thioether sulfone, and mixtures and combinations thereof.

2. The biomaterial of claim 1, wherein the biomaterial is in the form of a hydrogel.

3. The biomaterial of claim 2, wherein the linking groups comprise a linear or branched water soluble polymer or copolymer with at least two functional moieties chosen from maleimide, vinylsulfone, and combinations thereof.

4. The biomaterial of claim 3, wherein the water soluble polymer or copolymer comprises monomeric or copolymeric units chosen from poly(ethylene oxide) (PEO), poly(vinyl alcohol) (PVA), poly(acrylic acid) (PAA), polyacrylamide, poly(N-(2-hydroxypropyl) methacrylamide) (PHPMA), a polysaccharide, poly(ethylene glycol) (PEG), and combinations thereof.

5. The biomaterial of claim 4, wherein the water soluble polymer or copolymer comprises PEG.

6. The biomaterial of claim 3, wherein the water soluble polymer or copolymer comprises a multi-arm polymer.

7. The biomaterial of claim 3, wherein the water soluble polymer or copolymer comprises a biopolymer.

8. The biomaterial of claim 1, wherein the linking groups comprise thiosuccinimides derived from a thiol-ene reaction between thiol groups on the keratin proteins and a maleimide compound comprising a water-soluble polymer or copolymer.

9. The biomaterial of claim 1, wherein the linking groups comprise thioether sulfones derived from a thiol-ene reaction between thiol groups on the keratin proteins and a vinyl sulfone compound comprising a water-soluble polymer or copolymer.

10. The biomaterial of claim 1, wherein the keratin proteins are obtained from human hair.

11. The biomaterial of claim 10, wherein the keratin proteins comprise keratein.

12. The biomaterial of claim 1, wherein the biomaterial comprises a hydrogel comprising the keratin proteins crosslinked with one or more of the thiosuccinimide linking groups.

13. The biomaterial of claim 1, wherein the biomaterial comprises a hydrogel comprising the keratin proteins crosslinked with one or more of the thioether sulfone linking groups.

14. A method for making a biomaterial, the method comprising:
reacting a first aqueous pre-gel solution comprising a water soluble keratein with an aqueous solution comprising a crosslinking compound chosen from maleimide, vinyl sulfone, and mixtures and combinations thereof, to create the biomaterial comprising keratin proteins crosslinked with at least one of thiosuccinimide linking group or thioether sulfone linking group.
biomaterial comprising keratin proteins crosslinked with linking groups chosen from thiosuccinimide, thioether sulfone, and mixtures and combinations thereof.

15. A system for delivering components that react to form a biomaterial, the system comprising: at least one catheter configured to be disposed within a blood vessel, and wherein the at least one catheter is configured to deliver components that react to form, in the blood vessel and outside of the at least one catheter, the biomaterial comprising keratin proteins crosslinked with at least one of thiosuccinimide or thioether sulfone.

16. The method of claim 14, wherein the biomaterial comprises a hydrogel comprising the keratin proteins crosslinked with one or more of the thiosuccinimide linking group.

17. The method of claim 14, wherein the biomaterial comprises a hydrogel comprising the keratin proteins crosslinked with one or more of the thioether sulfone linking group.

18. The method of claim 14, wherein crosslinking compound comprises a linear or branched water soluble polymer or copolymer with at least two functional moieties chosen from the maleimide, the vinylsulfone, or combinations thereof.

19. The method of claim 18, wherein the water soluble polymer or copolymer comprises monomeric or copolymeric units chosen from poly(ethylene oxide) (PEO), poly(vinyl alcohol) (PVA), poly(acrylic acid) (PAA), polyacrylamide, poly(N-(2-hydroxypropyl) methacrylamide) (PHPMA), a polysaccharide, poly(ethylene glycol) (PEG), and combinations thereof.

20. The method of claim 19, wherein the water soluble polymer or copolymer comprises PEG.
